(19) United States
(12) Patent Application Publication
(10) Pub. No.: US 2017/0304593 A1
(43) Pub. Date: Oct. 26, 2017

(54) GUIDEWIRE TORQUER

(71) Applicant: Johan Willem Pieter Marsman, Hilversum (NL)

(72) Inventor: Johan Willem Pieter Marsman, Hilversum (NL)

(21) Appl. No.: 15/266,805

(22) Filed: Sep. 15, 2016

(30) Foreign Application Priority Data
Apr. 21, 2016 (NL) PCT/NL2016/050281

(51) Int. Cl. A61M 25/09

(52) U.S. Cl. CPC A61M 25/09041; A61M 2025/09116

(57) ABSTRACT

A guidewire torquer is described for manually controlling a guidewire, and imparting motion to a guidewire. The guidewire torquer comprises a handling body for handling of the guidewire torque. The guidewire torquer further comprises an insertion channel for receiving the guidewire. The insertion channel is arranged at a surface of the handling body, and the handling body comprises a shaping feature. The shaping feature provides tactile feedback as to the orientation of the guidewire, and the insertion channel provides a frictional fixation of the guidewire relative to the handling body.

### CROSS-REFERENCE TO RELATED APPLICATIONS

This application claims priority to PCT International Application No. PCT/NL2016/050281, filed Apr. 21, 2016, which is expressly incorporated by reference in its entirety, including the contents of any references contained therein.

### FIELD OF THE INVENTION

The present invention relates to a guidewire torquer.

### BACKGROUND OF THE INVENTION

A guidewire torquer is a device that is used in a number of different medical procedures to guide vascular catheters, catheter-mounted heart valves, aortic endografts, endotracheal tubes, or gastric feeding tubes and the like into a patient towards a desired location within the patient. Guidewires are used in a number of diagnostic and interventional fields, such as interventional cardiology, diagnostic and interventional radiology, vascular surgery, minimally invasive vascular interventions such as angioplasty, stenting, thrombolysis, transcatheter aortic valve insertion (TAVI), and endovascular abdominal aortic aneurysm repair (EVAR).

In vascular uses, a physician is required to navigate the guidewire through the vasculature of the patient. This is done in order to position the distal end of the guidewire at the desired location. Then a diagnostic or therapeutic catheter is fed over the guidewire to the desired location for the planned vascular intervention. In the text, the distal end of the guidewire is the end that is to enter the human body. The proximal end of the guidewire is in the hands of the physician and is not inserted into the body.

The distal end of the guidewire generally has an angled tip adjusted to help steer the guidewire. Positioning the distal end of the guidewire at the desired location can be tough and time consuming due to complex vascular anatomy and due to abnormalities of the vessel lumen caused by vascular disease. The physician manipulates the distal end of the guidewire through the vasculature of the patient to the desired location by pinching and torqueing the proximal end of the guidewire with his fingers.

Guidewires are relatively fine and difficult to grip between the physician's fingers, thereby making the positioning of the guidewire challenging. Handling of the guidewire is hampered by the fact that the guidewire is often slippery particularly when wetted with saline or blood. A guidewire is also intended to be smooth by means of various kinds of coating in order to provide lubricity between the guidewire surface and the inner surface of the vessel wall.

Due to the slipperiness of the guidewire the physician cannot accurately and securely rotate or move the guidewire lengthwise in and out of the body. It is also difficult to reliably feel with his fingers to what extent the guidewire follows his steering manipulations.

As such, a device called a guidewire torque device, guidewire torquer, or steering handle, is often affixed to the guidewire in order to allow the physician to better grip and impart motion to the guidewire. That is, the guidewire torquer device is intended to allow the physician to securely control the movements of the guidewire and to steer the distal end of the guidewire by rotational and longitudinal manipulation of the guidewire.

One disadvantage of prior art guidewire torquers is that they are configured to be attached from and over the proximal end of a guidewire. Generally, guidewire torquers must be back loaded over the proximal end of the guidewire, and then advanced along the guidewire until a suitable location is reached. After that a reliable fixation between guidewire and torquer is required, which may be unreliable or damage the guidewire by kinking or breakage or detachment of surface particles.

Furthermore, prior art guidewire torquers are complex, consisting of multiple moving parts, and therefore are relatively expensive.

### SUMMARY OF THE DISCLOSURE

In order to improve such prior art, the present invention provides a guidewire torquer for manually controlling a guidewire, and imparting motion to a guidewire in both lengthwise direction and rotational orientation, the guidewire torquer comprising:

a handling body for handling of the guidewire torquer,

an insertion channel for receiving the guidewire, the insertion channel being arranged at a surface of the handling body,

wherein the handling body comprises a shaping feature, which shaping feature provides tactile feedback as to the orientation of the guidewire, and

wherein the insertion channel provides a contact area of frictional fixation of the guidewire relative to the handling body.

A main advantage of such a guidewire torquer is that a superior and reliable manual control of the guidewire can be provided while the mounting of the guidewire torquer to the guidewire can be performed in a simple manner.

Also the mounting of the guidewire torquer to the guidewire can be performed at any spot of the guidewire as seen lengthwise to the guidewire, without the need for back loading the guidewire torquer over the proximal end of the guidewire.

Because of the frictional fixation of the guidewire inside the guidewire torquer, forces exerted by the guidewire torquer on the guidewire are spread over the contact area of frictional fixation. Therefore, the fixation is highly reliable. Also no movable parts are required to provide such friction.

Due to the fact, that forces exerted by the guidewire torquer on the guidewire are spread over the contact area, the following known problem of the prior art is prevented. In prior art devices clamping elements such as screws or claws pose a risk of detachment of particles from the coating of the guidewire, which is especially so in case of slippage of the guidewire relative to the torquer. Such damage, also known as local stripping of surface material or coating from the guidewire may pose health risks to patients being operated on, when such particles reach the inside of the human body.

Another aspect of possible damage by prior art devices to the guidewire is that a point force exerted by the guidewire torquer leads to deformation of the contour and shape of the guidewire, which could even lead to kinking or breakage of the wire. Such damages are prevented with the present guidewire torquer.

Also, the presented guidewire torquer provides a reliably visible and/or tactile feedback to the physician concerning the rotational orientation of the guidewire within the guidewire torquer, and a visible feedback concerning the insertion depth of the guidewire. According to the prior art such feedback to the physician is missing as the technical development of those devices has been directed solely at the capability to attach the device to the guidewire.

A first preferred embodiment according to the present invention provides a guidewire torquer wherein the insertion channel is accessible in a direction transverse to the channel over the whole length thereof. An advantage of said insertion channel is that the guidewire can be laid or pushed into the insertion channel over the whole length of the channel, allowing for easy sideways insertion of the guidewire into the insertion channel and therefore into the handling body of the guidewire torquer.

Further preferably, the insertion channel has a curved shape. The curved shape provides for an enhancement of the frictional fixation as the structural integrity of the guidewire provides pushing and pulling forces towards bends and inflection areas of the curved shape. The structural integrity of the guidewire provides the guidewire with an inclination to straighten out. Therefore, the guidewire will exert a force to an inner apex of a bend as well as to outer inflection points in between the apexes. Such forces enhance the friction between the guidewire torquer and the guidewire. The fact, that a guidewire is kept in the curved shape of the insertion channel, provides both a lengthwise and a rotational fixation of the guidewire to the handling body. The forces, provided by the heights of the bends in the insertion channel, exerted on to the guidewire make the guidewire rotating directly in response to any rotational handling of the guidewire torquer. This provides the physician with the ability to precisely manipulate the tip at the distal end of the guidewire.

Another preferred embodiment provides an insertion channel being shaped in the form of a sinusoid curve. Such curvature allows for a natural formation of curves and bends in the guidewire. The insertion channel may comprise one bend or multiple bends. For increasing friction, successive bends are preferably curved in opposite directions in an alternating fashion. The amount of friction between guidewire torquer and guidewire depends on the number and the amplitude of the curves (i.e. height of the bends). Generally, a higher bend provides in itself a higher friction than a lower bend. Furthermore, more bends exert more friction on the guidewire than less bends. Therefore, for the same magnitude of friction, multiple curves need less amplitude than a single curve.

An advantageously preferred embodiment provides a sinusoid insertion channel wherein the insertion channel is relatively wider at the apexes of the bends. Such local widening of the insertion channel facilitates the insertion of the guidewire into the insertion channel, without substantially changing effectiveness of the frictional fixation of the guidewire in the insertion channel.

According to a further preferred embodiment, the insertion channel exerts a friction force to the guidewire over a substantial portion of the length thereof, thereby preferably preventing point forces to the guidewire or pinching of the guidewire. Because the guidewire is making contact with the walls of the insertion channel over such large portion of the length of the insertion channel, the friction force is spread out over this portion. This provides a clear improvement compared to the highly focused or point friction forces of prior art devices.

A preferred embodiment, in which the insertion channel has an open side that is open to the side of the surface in which the channel is arranged, wherein the insertion channel has at least one portion in which, seen in cross-section, the open side of the insertion channel is narrower than a largest width of the insertion channel (in that cross-section), provides advantageously that the inwardly extending walls of the insertion channel urge the guidewire to stay in the insertion channel during operations.

It may also be provided that side walls of the insertion channel have an undercut portion only in areas where the guidewire is urged against the side wall, e.g. only in inner sidewalls in sections of the channel around the apex or apexes of curves and/or in outer sidewalls in sections of the channel at ends of U-shaped or (rounded) V-shaped sections of the insertion channel.

A further advantageous shape in this context is a trapezoidal cross-sectional profile of the insertion channel with the open side being narrower than the base of the insertion channel.

In order to improve tactile feedback as to the orientation of the guidewire torquer, the handling body preferably provides a flat or hollow surface at at least one side, wherein the handling body is preferably oval shaped or rectangular shaped with dimensions resembling those of a credit card. Also the shape preferably has bulges or indentations that further improve tactile feedback as well as improve the grip on the guidewire torquer by the fingers of the user. Holding such a device makes it easy to discern rotation during handling thereof. When the guidewire torquer is rotated, the degree of rotation is readily visible and tactile. Consequently, the degree of rotation of the guidewire that is fixated inside the guidewire torquer is visualized by the visible orientation of the shape of the guidewire torquer. Also, without looking at the guidewire torquer, the orientation of the hand holding the handling body provides tactile information as to the rotational orientation of the guidewire.

Further preferably, the insertion channel comprises a frictional surface arranged for increasing friction relative to the guidewire, such as a surface having a different texture and/or a surface of a different material than other surfaces of the torquer. The frictional surface may for instance be a course surface and/or a surface of a material having a high coefficient of friction such as rubber and the like. Such features according to this embodiment provide additional functionality as to the aspect of friction. There would be a higher requirement for such materials when the number and/or amplitude of the curves in the insertion channel is lower and vice versa. Clearly, a substantially straight insertion channel preferably comprises an exceptionally coarse surface in order to still build up sufficient friction between guidewire torquer and guidewire.

A highly advantageous embodiment provides the guidewire torquer being integrally formed of one part, preferably by means of a forming operation, such as injection molding or milling. Such production operation is highly reliable and cost-effective.

Another preferred embodiment provides two insertion channels, one on either side of the guidewire torquer, each insertion channel preferably having a different shape and/or frictional capacity so being optimized for different guidewires according to their thickness and/or stiffness.

In order to improve upon tactile orientation discernibility, a further preferred embodiment comprises a tactile marker, such as a top and/or bottom side indicator, that preferably comprises a coarseness at the surface of the guidewire torquer and/or a particular shape such as one or more indentations for the fingers of the user.

Further preferably, the guidewire torquer comprises a printable surface. An advantage thereof is that instructions and/or other information, such as relating to the use and origin of the guidewire torquer, can be provided also after the guidewire torquer has been unpacked and the packaging is dismissed.

According to a further preferred embodiment, at least one end of the insertion channel comprises a guidewire slip out prevention feature, preferably embodied as a relatively narrow open side of the insertion channel. Also an overhang of material at an insertion channel sidewall, opposite of and hanging over to the side of the nearest bend of the insertion channel may be provided. Such overhang is very effective in keeping the guidewire fixated in the insertion channel during operations.

Further preferably, the insertion channel comprises clickable portions at which the top side opening of the insertion channel is slightly narrower than the diameter of the respective intended guidewire. Thereby, the guidewire would click into the insertion channel with a sort of snapping operation when pushing it in through the slightly narrower open side of the insertion channel. Thus, the guidewire is forcibly retained in the insertion channel.

A further aspect according to the present invention provides a kit of a guidewire torquer according to one or more of the preceding claims with a guidewire, preferably a guidewire having an insertion tip with a curvature for inserting into the human body, the kit providing advantages as described in the above.

Preferably, in the kit, the guidewire also has an indicator tip at an opposite end of the guidewire compared to the end provided with the insertion tip for indicating the direction of the insertion tip as depending from the direction of the indicator tip. If between the insertion tip and the indicator tip the guidewire is in a straight condition, the insertion tip and the indicator tip are preferably curved to mutually opposite sides of the guidewire if the insertion channel ends point in generally the same direction (e.g. an U-shaped of V-shaped insertion channel) or to the same side if the insertion channel ends point in generally opposite directions (e.g. an S-shaped or Z-shaped insertion channel).

### BRIEF DESCRIPTION OF THE DRAWINGS

While the appended claims set forth the features of the present invention with particularity, further advantages, features and details of the present invention will be elucidated on the basis of a description of one or more embodiments with reference to the accompanying figures, of which:

FIG. 1 provides a perspective view of a first preferred embodiment according to the present invention.

FIGS. 2A, 2B, 2C and 2D provide four further views of the embodiment of FIG. 1.

FIGS. 3A, 3B, 3C and 3D provide a further preferred embodiment according to the present invention being mounted on a guidewire.

FIGS. 4A and 4B provide two front views of a further preferred embodiment according to the present invention.

FIG. 5 provides a perspective view of a further preferred embodiment according to the present invention.

### DETAILED DESCRIPTION OF THE DRAWINGS

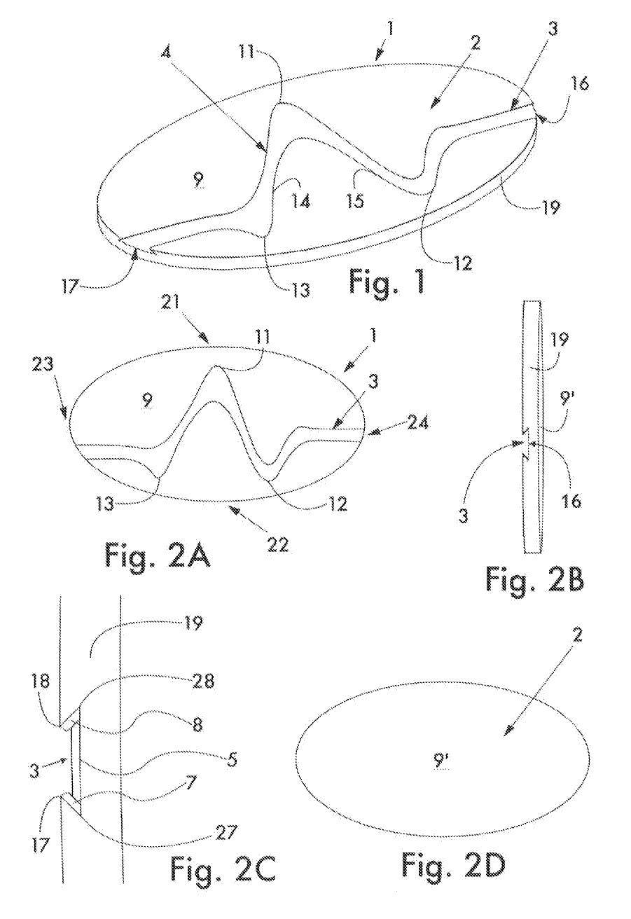

FIG. 1 shows a perspective view of a guidewire torquer 1 according to a first preferred embodiment according to the invention. FIGS. 2A-D show further views of the guidewire torquer 1. The guidewire torquer is comprised of a single handling body 2, which may be either molded or machined. The forming of the guidewire torquer from a single part provides a low complexity and expense of the device. The guidewire torquer 1 has the shape of a substantially flat, oval disc. The longitudinal diameter of the oval disc is about twice as long as the transverse diameter of the oval disc. The guidewire torquer 1 includes a front surface 9, a back surface 9′, a top side 21, a bottom side 22, a left side 23, a right side 24 and a circumference surface 19.

An insertion channel, score or groove 3 for fixing a guidewire relative to the guidewire torquer is present in the front surface 9 of the guidewire torquer. The insertion channel extends along the surface through the front surface and follows a curved stretch between the left side 23 and the right side 24 of the guidewire torquer. The insertion channel 3 trepans the circumference side 19 with a left opening 17 and with a right opening 16 and has a trapezoid profile.

The trapezoid profile is shown in greater detail in FIG. 2C. The base 5 of the insertion channel is bounded by two walls of the insertion channel, the lower wall 7 and the upper wall 8. The base 5 is connected with the lower wall 7 by the angular transition 27 and the base 5 is connected with the upper wall 8 by the angular transition 28. The lower wall 7 stretches between the angular transition 27 and the free edge 17 of the lower wall. The upper wall 8 stretches between the angular transition 28 and the free edge 18 of the upper wall. The open side of the insertion channel is bounded by the free edges 17 and 18. Due to the trapezoid profile of the insertion channel the width of the open side is smaller than the width of the base 5. The relatively narrow open side and the inclined walls provide urging of the guidewire towards the base of the insertion channel under the urging force of the wire itself being inclined to self-straighten.

The insertion channel 3 comprises a curved section 4 having one top bend 11 and two bottom bends 12 and 13. The guidewire is forced to follow the bends of the insertion channel resulting in friction between the guidewire and the insertion channel. Because of the friction the guidewire is firmly fixated relative to the handling body. Therefore, the guidewire torquer substantially becomes functionally one with the guidewire.

The insertion channel is wider at the bends than at the straight sections, facilitating the insertion of the guidewire into the guidewire torquer.

Furthermore, the insertion channel may be customized to be specific for selected guidewires in ways the skilled person would put the subject matter into practice based on this disclosure.

For the purpose of operation, the guidewire is inserted into the insertion channel of the guidewire torquer in a way similar to laying it into the channel following the bends thereby fitting the guidewire along the shape of the channel into the channel. Subsequently, the physician is able to control the rotational orientation of the guidewire and a tip at the distal end thereof. So the physician is able to steer and to navigate the guidewire through a vasculature of the patient. The physician is also able to both easily mount the guidewire torquer to the guidewire and to easily unlock the torquer from the guidewire. Therefore the user is able to quickly move the guidewire torquer from the one to the other location on the guidewire. These features are particularly useful by saving time for performing a procedure.

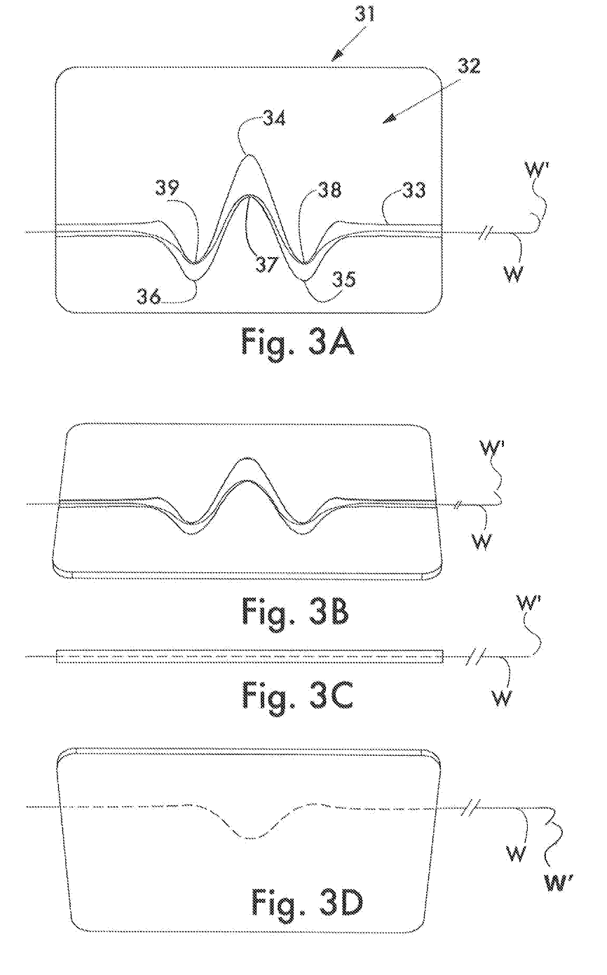

FIG. 3A shows a front view of a guidewire torquer 31. The guidewire torquer 31 is comprised of a handling body 32 and an insertion channel 33. Also a guidewire W with a tip W′ at its distal end is shown, resting in the insertion channel. The insertion channel 33 has a wavy or sinusoid shape having one top bend 34 and two bottom bends 35 and 36. Due to its natural stiffness the guidewire reaches the position in which the guidewire W firmly touches the inner apexes 37, 38 and 39 of the bends in the insertion channel 33.

Due to the resulting friction between the guidewire W and the guidewire torquer 31 the guidewire substantially becomes functionally one with the guidewire torquer.

Therefore, as is illustrated in the FIGS. 3B to 3D, the rotational orientation of the guidewire in the insertion channel is precisely defined by the rotational orientation of the guidewire torquer.

Compared to FIG. 3A, the guidewire torquer 31 has been tilted 45 degrees backwards in FIG. 3B, resulting concurrently in a similar change in rotational orientation of the distal tip W′ backwards over 45 degrees. Likewise, in the FIGS. 3C and 3D a backwards tilt of the guidewire torquer over 90 and 135 degrees directly causes a corresponding backwards rotation of the distal tip W′ over 90 and 135 degrees.

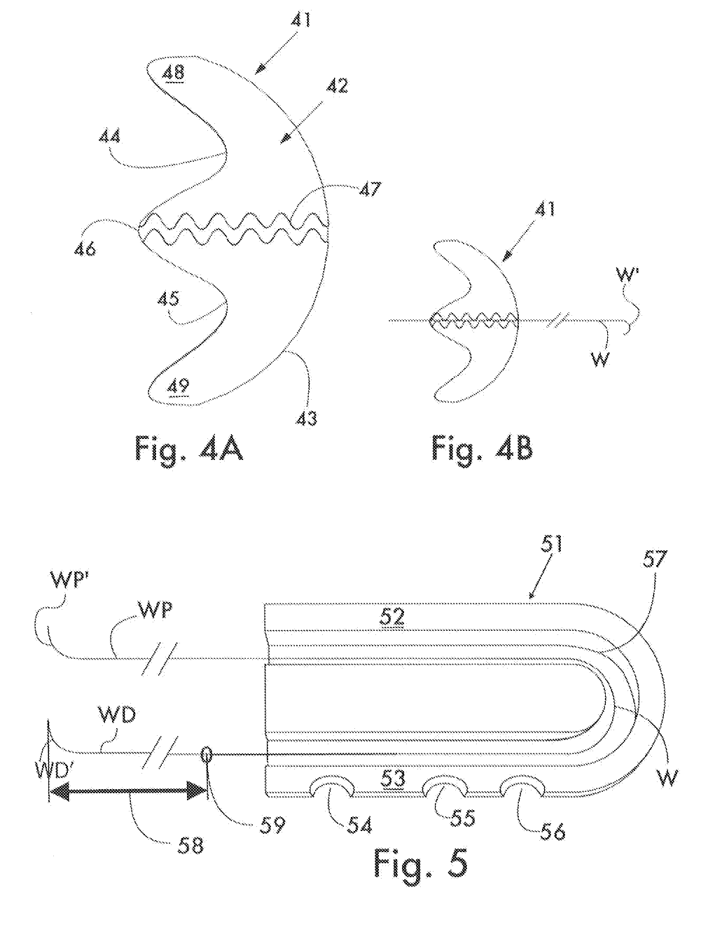

FIG. 4A shows a further preferred embodiment of a guidewire torquer 41 with a substantially flat handling body 42 in a shape having a convex right side 43 and a left side with 2 recesses 44, 45 and a protrusion 46 in between. An insertion channel 47 with multiple low amplitude bends extends from the protrusion 46 to the convex right side 43 of this embodiment. The top side 48 and the bottom side 49 of the guidewire torquer 41 are symmetrically and widely spaced from the insertion channel 47. FIG. 4B shows the guidewire torquer 41 mounted on a guidewire W whose distal tip W′ has a curved shape.

Functionally, the configuration of this embodiment provides the guidewire torquer 41 with a stable position when positioned on a flat surface. A rotational force exerted to the guidewire torquer, transmitted to the torquer e.g. by the guidewire W, will not result in rotation of the guidewire torquer. Therefore, withstanding the exerted rotational force, the guidewire torquer 41 will remain unchanged in its stable position relative to the flat surface it has been laid upon. As described in the above, when a guidewire torquer of the present invention is mounted on a guidewire, together they form one functional unit due to the firm fixation of the torquer relative to the guidewire. Therefore, the embodiment shown in FIG. 4, as well as other embodiments, serves as a stabilizer relative to the surface it is laid upon of the rotational orientation both of the guidewire torquer 41 and of the guidewire W.

Moreover the purpose of this embodiment is to keep the tip W′ of the guidewire reliably pointing to a specific desired direction during advancement of the guidewire in the human body. In FIG. 4B the tip W′ is directed into the same direction as the bottom side 49 of the guidewire torquer 41, which direction is downwards. Due to its substantially flat shape the guidewire torquer 41 will remain in parallel position to the surface it is laid upon when it is dragged to the right over the surface by the guidewire, in a procedure where the physician advances the tip W′ of the guidewire further into the human body.

An example of such procedure is a placement of a central vein catheter (CVC). The CVC is fed over the guidewire W which tip W′ first has been navigated from the subclavian vein into the central vein. During the navigation of the guidewire to the central vein the guidewire torquer 41 keeps the tip W′ of the guidewire constantly directed downwards, that is into the direction of the central vein. Consequently, the tip W′ preferentially enters the central vein. So, the function of the guidewire torquer 41 is to allow for the correct placement of the guidewire W in the central vein, and therefore to allow for the correct placement of the CVC that is fed over it.

By doing so the guidewire torquer 41 prevents the tip W′ of the guidewire to turn upwards and erroneously enter the jugular vein, resulting in the CVC erroneously being fed over the guidewire into the jugular vein as well. Such misplacement of the CVC is the most frequent complication of CVC placement procedures.

FIG. 5 provides a preferred embodiment in which a guidewire torquer 51 has a thicker top side 52 than a bottom side 53. This asymmetry provides the user tactile information as to the position of the top side 52 relatively to the position of the bottom side 53 of the guidewire torquer 51. The bottom side 53 has three indentations 54, 55 and 56, that allow for further improved tactile feedback as well as for a firm grip on the guidewire torquer by fingers of the physician when feeling the indentations or placed therein.

The guidewire torquer 51 has an insertion channel 57 that is substantially shaped like a U-turn. Therefore, a proximal portion WP of a guidewire W that has been inserted in the U-turn shaped insertion channel 57 points into the same direction as a distal portion WD of the guidewire W. The proximal portion WP has a proximal tip WP′ with a shape that resembles the shape of the distal tip WD′.

A combination of the guidewire W and the guidewire torquer 51 provides the physician with information as to both the rotational orientation and the insertion depth of the distal end WD′ of the guidewire W. In the text the insertion depth of a guidewire measures a distance from a distal end of the guidewire located inside the human body to a point of entry of the guidewire into the patient. In FIG. 5 the insertion depth of the guidewire W measures the distance 58 between the distal end WD′ and the point of entry 59 of the guidewire into the human body. Without any measurement the insertion depth of the guidewire W is visually indicated by the position of the proximal tip WP′, whenever the guidewire has been positioned in the insertion channel in a symmetrical way relatively to the curve of the U-turn insertion channel, which is the case in FIG. 5.

Using the guidewire torquer 51 can be highly advantageous because it allows the physician to substantially reduce screening time and therefore the procedural radiation exposure. Indeed, feedback that is provided by the guidewire torquer 51 about the rotational orientation and the insertion depth of the guidewire is available for the physician without the need for additional fluoroscopy. Obtaining such information without the said feedback inevitably requires screening of the guidewire inside the patient, thereby increasing the amount of radiation exposure and prolonging the procedure time. In contrast, the rotational orientation and the insertion depth of the distal tip of the guidewire inside the human body can be easily and continuously derived from the rotational orientation and position of the proximal tip that is readily visible outside the human body.

The present invention is described in the foregoing on the basis of several preferred embodiments. Different aspects of different embodiments can be combined, wherein all combinations which can be made by a skilled person on the basis of this document must be included. These preferred embodiments are not limitative for the scope of protection of this document. The rights sought are defined in the appended claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A guidewire torquer for manually controlling a guidewire and imparting motion to a guidewire, the guidewire torquer comprising:
a handling body for handling of the guidewire torquer, and
an insertion channel for receiving the guidewire, the insertion channel being arranged at a surface of the handling body,
wherein the handling body comprises a shaping feature, which shaping feature provides visible and/or tactile feedback as to the orientation of the guidewire, and
wherein the insertion channel provides a frictional fixation of the guidewire relative to the handling body.

2. A guidewire torquer according to claim 1, wherein the insertion channel is accessible in a direction transverse to the channel over the whole length thereof.

3. A guidewire torquer according to claim 1, wherein the insertion channel has a curved shape.

4. A guidewire torquer according to claim 1, in which the insertion channel forms a curved surface channel in the handling body.

5. A guidewire torquer according to claim 1, the channel being shaped in the form of a sinusoid curve.

6. A guidewire torquer according to claim 1, the channel having one single bend.

7. A guidewire torquer according to claim 1, the channel comprising at least two bends.

8. A guidewire torquer according to claim 1, further comprising two or more insertion channels, each channel having a different shape and/or frictional capacity.

9. A guidewire torquer according to claim 1, further comprising two or more insertion channels, at least one of said insertion channels being arranged on a front side of the guidewire torquer and at least another one of said insertion channels being arranged on a back side of the guidewire torquer.

10. A guidewire torquer according to claim 1, wherein the insertion channel is arranged for exerting a friction force to the guidewire over a substantial part of the length thereof.

11. A guidewire torquer according to claim 1, the insertion channel further comprising a frictional surface arranged for increasing friction relative to the guidewire.

12. A guidewire torquer according to claim 1, in which the insertion channel has an open side that is open to the side of the surface in which the channel is arranged, wherein the insertion channel has at least one portion in which, seen in cross-section, the open side of the channel is narrower than the a largest width of the channel.

13. A guidewire torquer according to claim 1, in which the insertion channel has a substantially trapezoid cross-sectional profile.

14. A guidewire torquer according to claim 1, in which at least one end of the insertion channel comprises a guidewire slip out prevention feature, embodied as a relatively narrow open side of the insertion channel or an overhang of material at an insertion channel sidewall, opposite of and hanging over to a side of the nearest bend of the insertion channel.

15. A guidewire torquer according to claim 1, in which the insertion channel comprises clickable portions at which the top side opening of the channel is slightly narrower than the diameter of a respectively intended guidewire.

16. A guidewire torquer according to claim 1, in which the handling body comprises a substantially flat or hollow surface at at least one side for providing a rotationally stable position when placed on a flat surface.

17. A guidewire torquer according to claim 1, in which the handling body comprises a substantially flat shape for providing a rotationally stable position when moved over a flat surface.

18. A guidewire torquer according to claim 1, further wherein the handling body has a flat or hollow surface at at least one side.

19. A guidewire torquer according to claim 1, wherein the handling body has bulges and/or indentations.

20. A guidewire torquer according to claim 1, the guidewire torquer being made of one part.

21. A guidewire torquer according to claim 1, further comprising a bottom and/or top side indicator.

22. A guidewire torquer according to claim 1, further comprising a tactile marker.

23. A kit comprising:
a guidewire; and
a guidewire torquer for manually controlling a guidewire and imparting motion to a guidewire, the guidewire torquer comprising:
a handling body for handling of the guidewire torquer, and
an insertion channel for receiving the guidewire, the insertion channel being arranged at a surface of the handling body,
wherein the handling body comprises a shaping feature, which shaping feature provides visible and/or tactile feedback as to the orientation of the guidewire, and
wherein the insertion channel provides a frictional fixation of the guidewire relative to the handling body.

24. A kit according to claim 23, wherein the guidewire has an insertion tip with a curvature for inserting into the human body.

25. A kit according to claim 24, the guidewire further having an indicator tip at an end of the guidewire opposite of the insertion tip, wherein, if between the insertion tip and the indicator tip, the guidewire is in a straight condition, the insertion tip and the indicator tip are curved to a common side of the guidewire or to mutually opposite sides of the guidewire for indicating the direction of the insertion tip as depending from the direction of the indicator tip.
